(19) United States
(12) Patent Application Publication
(10) Pub. No.: US 2015/0231351 A1
(43) Pub. Date: Aug. 20, 2015

(54) SYSTEM FOR OPTIMAL MECHANICAL VENTILATION

(71) Applicant: INNOTEK AB, Lund (SE)

(72) Inventor: Björn Jonson, Lund (SE)

(21) Appl. No.: 14/430,172

(22) PCT Filed: Oct. 22, 2013
(86) PCT No.: PCT/SE2013/000162

§ 371 Date: Mar. 20, 2015

(30) Foreign Application Priority Data
Sep. 24, 2012 (SE) 1230100-8

(51) Int. Cl. A61M 16/00; A61M 16/08

(52) U.S. Cl. CPC A61M 16/0069; A61M 16/0003; A61M 16/085; A61M 2016/003; A61M 2016/0027; A61M 2016/0015; A61M 2205/3334; A61M 2230/432; A61M 2205/60

(57) ABSTRACT

The invention relates to a system for mechanical ventilation comprising transducers for measurement of airway flow rate, pressure and CO₂ and at least one computer that records and analysis the transducer signals. The operator defines physiological specified goals or accepts default values. Specified physiological goals relate to CO₂ exchange and to volumes and pressures so as to minimise deleterious effects of ventilation. On the basis of physiological information about the respiratory system characterised according to the principle of volumetric capnography and lung mechanical parameters, the computer performs analytical calculations in order to identify one or more modes of ventilator operation leading to specified goals. Such a mode of operation is implemented manually or automatically in one or more steps. The physiological outcome of resetting is reported and an alarm is issued when the outcome deviates from expectations. The computer may perform repeated automatic measurements and repeat the resetting in order to reach and maintain a status of the patient coherent with specified goals.

### BACKGROUND OF THE INVENTION

1. Field of the Invention

The present invention relates to an apparatus and a method used at mechanical ventilation of man or animal, hereafter referred to as the patient, in which a computer performs a mathematical analysis of the transducer signals to identify a mode of ventilation that is optimal with respect to current specified physiological goals. After an analysis of the physiological properties of the respiratory system the changes in current mode of ventilator operation which should be performed in order to come closer to the goals are calculated. After implementation of such changes the computer may check that changes in physiological status are in direction of the goals. The computer may also be programmed for automatic ventilator resetting.

2. Description of the Prior Art

The properties of the respiratory system comprising airways, lung parenchyma, alveoli, pulmonary blood vessels, heart and thoracic cage are complex, particularly so in disease. The operator of a ventilator, usually a physician or a respiratory therapist, frequently changes the setting of a ventilator. The purpose behind resetting is to reach desired goals of mechanical ventilation. Due to the complexity of physiology, it is in general not possible to foresee which effects resetting will have on respiratory mechanics, gas exchange and circulation, particularly when resetting comprises several parameters defining the mode of ventilation.

Ventilation serves the purpose of gas exchange between the respired air and the pulmonary capillary blood and indirectly the arterial blood. Adequate oxygenation can be achieved at low degrees of alveolar ventilation by using high oxygen fractions of inspired gas. In contrast, proper elimination of CO₂ requires that alveolar ventilation matches the metabolic production of CO₂. For control of the efficiency of ventilation, a particularly important parameter is arterial partial pressure of carbon dioxide, PₐCO₂, that reflects alveolar ventilation and influences arterial pH. The body contains large amounts of exchangeable carbon dioxide, CO₂. This implies that a change of alveolar ventilation leads to a slow change in PₐCO₂. It takes more than 20 minutes after ventilator resetting to reach a new steady state with regards to PₐCO₂ and arterial pH. Accordingly, an arterial sample does not properly indicate the effect on PₐCO₂ until long after resetting. During that interval changes in the physiological status of the patient may occur, which may obscure the effect of resetting.

When ventilator resetting is left to an operator's considerations about which might be a proper mode of operation there is a need for fast feedback to the operator, implying that information is gained allowing judgement of which results ventilator resetting has on the patient. A system for such feedback is described in the Swedish patent application, SE1200155-8 of 2012. The objective of this former invention is to identify beneficial and adverse effects of ventilator resetting within few breaths. Monitored parameters include e.g. tidal volume, airway pressures, end tidal CO₂, hemodynamics and volume of CO₂ eliminated per minute, V_{MIN}CO₂. Values of such parameters before and after ventilator resetting are presented to the operator. The quotient between V_{MIN}CO₂ after and before resetting is used to illustrate the effect on alveolar ventilation and thereby the effect on PaCO₂. This former invention is used in conjunction with ventilator resetting based upon the operator's judgement and opinion about which alternative setting that should be beneficial. A limitation of the former invention is that the system does not provide any assistance with respect to how the ventilator should be reset in order to attain the physiological goals of the resetting.

A method that may alleviate problems to foresee effects of resetting a ventilator is computer simulation of ventilator resetting. Such simulation is based upon a physiological profile of the respiratory system that is defined with a computer program that measures and analyses the physiological properties of the respiratory system prior to the simulation. The simulation relies upon a physiological model and a mathematical description of the function of a ventilator at different settings. Such a method is described in U.S. Pat. No. 6,578,575 B1. The simulation may be of such nature that it continues until a mode of ventilator operation has been identified, which would lead to goals defined by the operator. Simulation of a number of parameters defining mode of ventilator operation leads to multiple degrees of freedom in the simulation causing instability in the process. This may end up in a false minimum of error. Accordingly, there is a need for alternative systems.

An invention described in the British patent 1 581 482 from 1980 is based upon that the mode of operation of a ventilator is at times of a “physiological test” deliberately disturbed and that the effect of this disturbance is used to automatically control the ventilator. The nature of disturbance mentioned is short periods of change in composition of inhaled gas. In this patent the nature of the “disturbance” is not based upon an analysis such that the disturbance itself leads towards physiological goals.

An invention described in the patents U.S. Pat. No. 6,709,405 and EP1295620 is based upon a system with capacities similar to those of a ServoVentilator 900 C complemented by an external computer that through the socket for external control of ventilator function takes over the control of the ventilator. Such a system can be used to perform physiological tests in order to provide detailed information about mechanics and gas exchange of the respiratory system. Even in these patents, there is no indication of any method according to which physiological observations are analysed in order to change the mode of operation of a ventilator so as to reach specified physiological goals.

Adequate gas exchange with respect to CO₂ and O₂ is a primary goal behind mechanical ventilation. Other goals are related to protection of the lungs against ventilation induced lung injury, VILI, and other adverse effects for example on blood circulation and heart function. High tidal volumes and high airway pressures are injurious to the lung and must be controlled to avoid VILI. In the acute respiratory distress syndrome, ARDS, repetitive lung collapse and re-expansion of lung units is a particularly injurious process that must be avoided. In chronic obstructive pulmonary disease, COPD, hyperinflation leading to lung damage and perturbation of circulation should be mitigated.

Setting of a ventilator entails a large number of physiological and therapeutic aspects and many parameters defining the mode of ventilator operation to be set. The principle mode of ventilation may be volume or pressure controlled or combinations of these two. Many forms of supported spontaneous breathing exist. For each chosen principle mode of ventilation a large number of parameters can be set in order to reach physiological and therapeutic goals.

### Groups of Set Parameters

A modern ventilator allows great variation of its mode of operation. In the present context mode of operation denotes all aspects from volume or pressure controlled ventilation, to assisted ventilation and to details of each of those like respiratory rate, RR, tidal volume, V_{T}, inspiratory pressure and others. Set, setting or resetting refer in this document to both manually and by computer effectuated modes of ventilation. Parameters set on the ventilator may be grouped according to which principle effect each parameter has on ventilation.
1. At volume controlled ventilation, RR and V_{T} together define minute ventilation.
2. At pressure controlled ventilation, minute ventilation is determined by RR and the difference between inspiratory pressure and positive end expiratory pressure, PEEP, together with compliance of the respiratory system.
3. Pattern of inspiratory gas delivery entails inspiration time, T_{I}, postinspiratory pause time, T_{P}, and shape of the inspiratory wave form leading to constant, decreasing or increasing flow rate influence together gas mixing in the lungs and thereby dead space and CO₂ exchange.
4. The value of set positive end expiratory pressure, PEEP, influences volume and pressure levels around which tidal ventilation occurs.

The rough subdivision of some selected parameters under point 1-4 above serves as a basis for the description of the invention. Points 1-3 are central for gas exchange, particularly that of CO₂. PEEP is an important parameter with respect to lung protective ventilation and in large patient groups also for oxygenation of blood in the lungs.

As will be evidenced below, even the most experienced operator cannot select the most optimal combination of all parameters. For less trained operators responsible for patient care day and night, the problems are overwhelming.

In order to approach reasonable settings, guidelines are offered for some particular situations. The most well known treatment protocol is for ARDS. This is based upon an article from the ARDSnet¹ and aims at lung protection by using a tidal volume ≦6 ml/ideal body weight and a postinspiratory plateau pressure, P_{PLAT}, of ≦30 cmH₂O, Oxygenation is maintained by choosing combinations of fraction of inspired oxygen, F_{I}O₂, and PEEP, according to a table. Although this strategy is advantageous relative to outdated treatment with high tidal volumes and high P_{PLAT} it cannot be optimal for the individual patient because the large variation in physiology between patients is not taken into account. Furthermore, increasing evidence talks in favour of even lower tidal volumes than 6 ml/kg. For other diseases like COPD, the knowledge about adequate ventilation patterns is even less than for ARDS and current recommendations are based on obsolete theories.

### The Invention

The present invention can be practised at different principle modes of operation like volume and pressure controlled ventilation, combinations between these and also supported ventilation. The invention can be practised at all diseases and even when lungs are healthy.

The objective of the present invention is to aid the operator to find a mode of ventilator operation which is optimal with respect to goals related to physiological effects of ventilation. Goals depend on the category of the actual patient. For example; goals for patients with brain damage may be modest hypocapnia at lowest feasible mean airway pressure. Goals at ARDS may be to maintain normocapnia or modest hypercapnia, by using a tidal volume as low as possible and at a lung protective value of P_{PLAT}. This implies that PEEP will be as high as is compatible with adequate CO₂ exchange and lung protective values of V_{T} and P_{PLAT}. Thereby, high PEEP will keep the lung open and provide optimal conditions for oxygenation. High PEEP is lung protective by allowing an optimally low value of F_{I}O₂ and by preventing expiratory lung collapse. In COPD, mechanical ventilation is practised mainly in life threatening situations. Goals are to alleviate hyperinflation and hypercapnia, which are major problems caused by extremely high expiratory airway resistance.

The system has sensors for measurement of CO₂ concentration, flow rate and pressure in the airway and samples the signals at a rate high enough to allow detailed analysis of CO₂ exchange and respiratory mechanics. Sensors and a computer performing such analysis may be integrated with the ventilator into one single apparatus. An alternative is that the sensors and the measuring and analysing computer are auxiliary to the ventilator. Several alternative configurations are possible, two of which are depicted in FIGS. 1 and 2. Signals representing circulation, such as arterial pressure, are commonly recorded by auxiliary monitoring equipment. According to preferred embodiments of the invention, information from these may be fed to the system.

The system calculates and monitors parameters representing gas exchange, airway pressure and others, which are relevant with respect to the defined goals. Examples of such parameters are: Volume of CO₂ eliminated by ventilation per breath, V_{T}CO₂, or per minute, V_{MIN}CO₂, fraction of end-tidal CO₂, F_{eT}CO₂, V_{T}, RR, P_{PLAT}, PEEP, and total PEEP, PEEP_{TOT}, i.e. PEEP set on the ventilator plus auto-PEEP. Additional parameters reflecting circulation, for example arterial pressure may be recorded and analyzed. Oxygenation is monitored by saturation in blood measured in the periphery, S_{P}O₂, alternatively as partial pressure of oxygen using an indwelling sensing catheter.

### DESCRIPTION OF THE DRAWINGS

FIG. 1

FIG. 1 illustrates a ventilator 1 that accords with a preferred embodiment of the invention. The apparatus is only schematically depicted, since with modern technology, configuration options are virtually unlimited.

A pneumatic inspiratory system of the ventilator comprises inlets for gases like air and oxygen 2, a blender for the gases 3 and a flow controller in the inspiratory line 4. In an alternative embodiment of the invention the blender 3 and the controller 4 are integrated into a single unit. The inspiratory line is equipped with a flow meter 5. Outside the ventilator or integrated into the ventilator the inspiratory line is often equipped with a humidifier 6 and continues in the form of a flexible inspiratory tube 7 that leads to the Y-piece 8. The ventilator is connected to the patient 10 with a tracheal tube 9 but can be connected by other means. Expiration occurs through a pneumatic expiratory system of the ventilator starting at the Y-piece 8 and further through a flexible expiratory tube 11, an expiratory valve 12 and an expiratory flow meter 13. The order of 12 and 13 may be the opposite. A CO₂ analyzer 14 measures fraction of CO₂ at the Y-piece. A pressure transducer 15 measures airway pressure. It can alternatively be connected to the expiratory line 11 or be duplicated in both inspiratory line and in expiratory line. The function of the ventilator is controlled by an electronic control unit 17 that may be an analogue or digital device. In a preferred embodiment of the invention the control unit comprises at least one computer that records and analyzes the signals from flow, pressure and CO₂ transducers 5, 13, 15 and 14. This computer can also receive signals from systems for monitoring of circulation such as arterial pressure and S_{P}O₂. The control unit is able to communicate with the user through a keyboard, by touch controls or by other means. Communication is also possible from distance, e.g. from a central system in a critical care unit. All the stipulated parts can be integrated into a single apparatus or functionally distributed among different physical units. The latter option could mean that e.g. the function serving to control the pneumatic systems is located within the ventilator, whereas e.g. calculation and monitoring functions are physically located in another apparatus such as an external computer.

The control unit receives analogue or digital signals representing flow rate, pressure and CO₂ and sends signals to the inspiratory and expiratory valves 4 and 12 through means for electronic communication 16. The control unit may apart from components within the ventilator itself comprise components and systems outside the ventilator. The technique of today offers virtually limitless possibilities to embody the invention with respect to technical solutions of electronic components and their communication with each other by wired or wireless means. Monitoring and analysis of the ventilation process may be achieved by a system incorporated in the ventilator or by a system outside the ventilator. The control unit 17 is in a preferred embodiment of the invention equipped with a visual screen for monitoring of flow and pressure signals and for display of other information.

FIG. 2

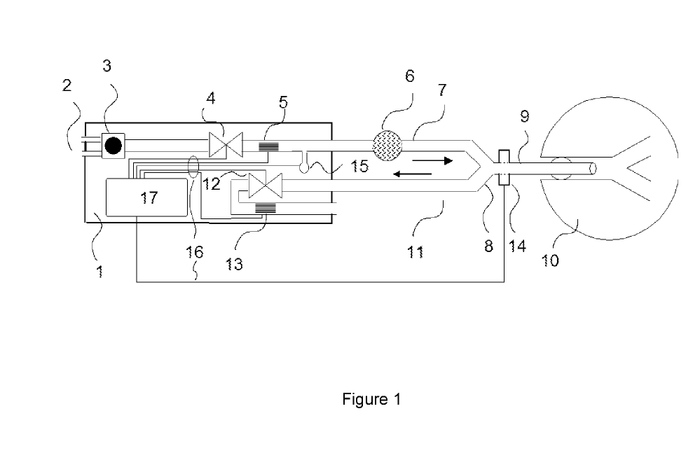

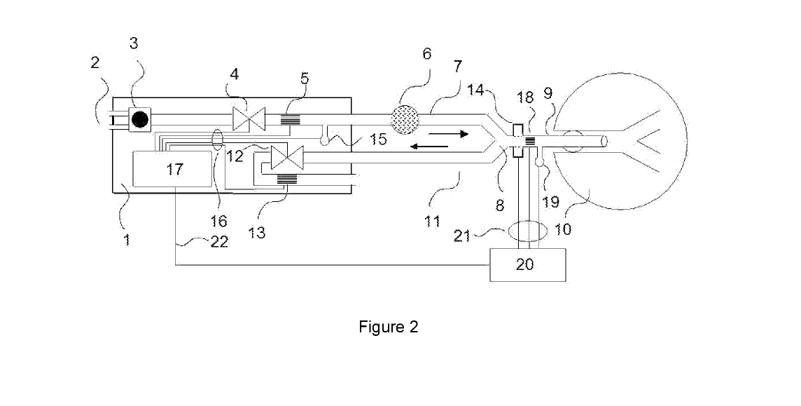

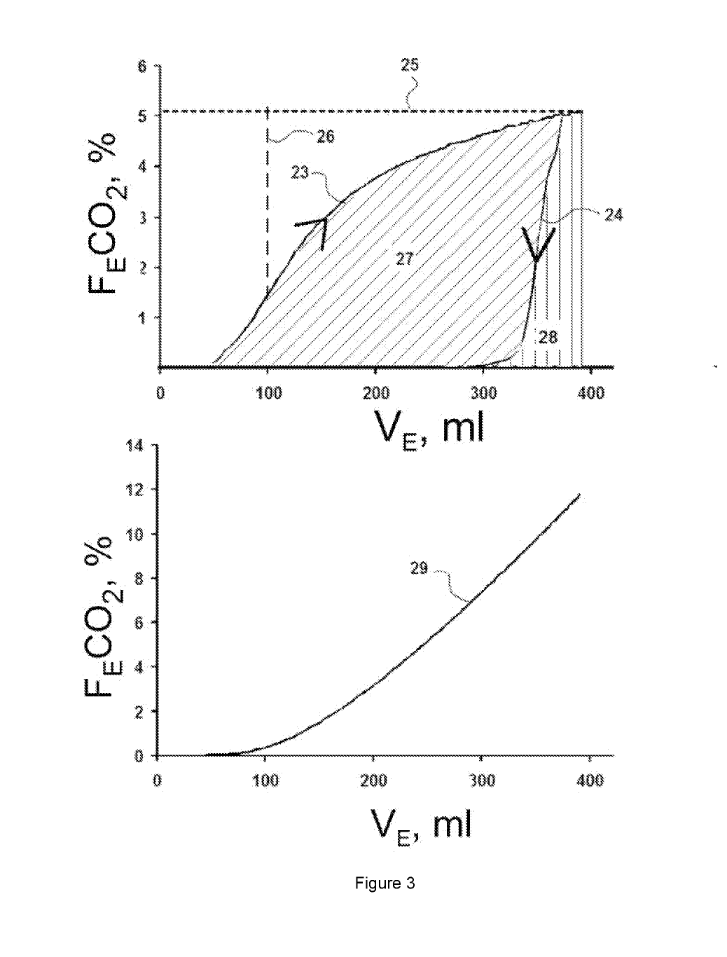

FIG. 2 illustrates an alternative preferred embodiment of the invention in which the numbers 1-17 indicate the same structures as in FIG. 1. The system used for monitoring according to the present invention is embodied within an apparatus that is separate from the ventilator 1. The monitoring apparatus comprises a computer 20 and transducers for CO₂ 14 flow rate 18 and airway pressure 19, which through wired or wireless means of communication 21 send signals to the computer 20. According to a further embodiment of the invention not shown in FIG. 2, the computer 20 may receive signals for one or more of the parameters flow rate, airway pressure and CO₂ from transducers integrated in the ventilator thus avoiding duplication of transducer equipment. The computer 20 may also have access to other information from the ventilator 1 such as ventilator setting, respiratory rate and information about timing of partitions of the respiratory cycle through a digital or analogue, wired or wireless communication link 22. Likewise, the computer 20 may receive information from other sources such as those used for monitoring of circulation like arterial pressure. According to a preferred embodiment of the invention, the computers 20 and 17 may be linked so as to exchange information with each other in analogue or digital format through a wired of wireless communication system 22. The computer 20 can thereby send signals to computer 17. Such an embodiment may allow the computer 20 to modify the mode of operation of the ventilator 1.

The transducer for CO₂ must not be placed at the y-piece as shown in FIGS. 1 and 2. It may be placed in the pneumatic expiratory line 11.

FIG. 3

Analysis of CO₂ elimination and its dependence upon V_{T} and other parameters describing ventilation is based upon the principles of volumetric capnography in the form of the single breath test for CO₂, SBT-CO₂, illustrated in FIG. 3. In FIG. 3 upper panel, the fraction of CO₂ in expired gas, F_{E}CO₂, recorded in an ARDS patient is shown by the rising limb 23 plotted against expired volume, V_{E}. Fraction of CO₂ in re-inspired gas is shown by the falling limb 24. F_{eT}CO₂ is indicated by the interrupted horizontal line 25. Airway dead space, V_{Daw}, illustrated by interrupted vertical line 26 can be estimated according to several known algorithms.

V_{T}CO₂ corresponds to the diagonally hatched area 27. The volume of CO₂ re-inspired at the start of inspiration, V_{I}CO₂, is represented by the vertically hatched area 28. The volume of CO₂ exhaled during expiration, V_{E}CO₂ corresponds to the combined hatched areas. V_{T}CO₂ may be measured as the difference (V_{E}CO₂−V_{I}CO₂). V_{I}CO₂ may be measured from the SBT-CO₂ or may be estimated by other means for example from the value of F_{eT}CO₂ in combination with known properties of the tubing system. The latter embodiment of the invention is applied when the transducer for CO₂ is not placed at the y-piece 8 but in the pneumatic line 11, because at such an embodiment V_{I}CO₂ cannot be measured. Under circumstances under which V_{I}CO₂ is negligible V_{T}CO₂ may be considered equal to V_{E}CO₂. This is the case when the y-piece and nearby tubing is flushed free from CO₂ before inspiration.

An alternative presentation of the expiration limb of the SBT-CO₂ 23 is V_{E}CO₂ related to V_{E}, FIG. 3 lower panel. This curve 29 is obtained by integration over time of the product (flow rate·F_{E}CO₂).

### DESCRIPTION OF PREFERRED EMBODIMENTS

The system is based upon sensors for airway flow rate, pressure and CO₂ as illustrated in FIGS. 1 and 2. Flow rate and airway pressure may be measured within the ventilator 5, 13 and 15 in FIG. 1 or at the airway opening of the patient 18, 19 in FIG. 2. For all embodiments of the invention, signals for flow rate, airway pressure and CO₂ should have an adequate frequency response and be adequately in synchrony with each other so that events during breaths representing each signal or combinations of signals can be accurately recorded and monitored. Optional transducers for S_{P}O₂, arterial pressure and other signals are foreseen to be incorporated in alternative embodiments of the invention.

A computer that may be integrated into the ventilator 17 or be a separate computer 20 samples the signals for CO₂, airway pressure and flow at an adequate rate. These signals, together with data emanating from analyses of the signals and other information may be displayed and stored by the computer in accordance with conventional monitoring systems. Accordingly, volumes are calculated by integration of flow rate over time. RR may be derived from signals controlling the valves of the ventilator 4, 12 or from analysis of pressure and flow signals by the computer 17 or 20.

The signals representing airway CO₂ concentration, flow rate and pressure are analysed with respect to gas exchange with focus on CO₂ turnover and mechanics of the respiratory system so as to predict the results of ventilator resetting. Notably, in the following ventilator resetting refers to a change in the mode of operation of the ventilator that may follow from manual or automated action.

The invention is based upon analytical mathematical calculations of how alternative modes of ventilation would affect the physiological status of the patient. The purpose is to identify a mode based upon ventilator resetting that leads to specified goals. A specified goal may be a specific value of a parameter or a range of values below or above a specific number. The analysis starts with analysis of CO₂ exchange in relation to subdivisions 1-3 of parameters set on the ventilator and continues with PEEP and other parameters, which influence volume and pressure levels around which tidal ventilation occurs.

### Analysis of CO2 Exchange

Recorded flow and CO₂ values are analyzed according to the SBT-CO₂, FIG. 3. In a physiological steady state PaCO₂ reflects the quotient between metabolic production of CO₂ in the body and efficient alveolar ventilation, both measured as volume per minute. After a sudden change in alveolar ventilation caused by ventilator resetting, PaCO₂ will change in proportion to the change in alveolar ventilation but in opposite direction. Because of large CO₂ stores in the body, the change in PaCO₂ occurs slowly. It takes at least 20 minutes to reach a new steady state. However, immediately after the resetting, V_{MIN}CO₂ changes in direct proportion to the change in alveolar ventilation. This change can be observed during a short period of time before CO₂ stores have significantly changed. The period is approximately 1 minute. Later on, V_{MIN}CO₂ returns towards the value corresponding to metabolic CO₂ production.

After ventilator resetting an upcoming value of PaCO₂ may be calculated from the current values of PaCO₂ and V_{MIN}CO₂ and V_{MIN}CO₂ predicted to occur within about one minute after resetting.

PaCO_{2new}=PaCO_{2current}·(V_{MIN}CO_{2current}/V_{MIN}CO_{2new}) Eq. 1

In Eq. 1 and in the following subscripts ‘current’ and ‘new’ indicate values before and after ventilator resetting, respectively. Notably, PaCO_{2new} refers to a new steady state while V_{MIN}CO_{2new} refers to data immediately after resetting.

According to the present invention, a change in PaCO₂ after ventilator resetting is calculated from measured value of V_{MIN}CO_{2current} and a predicted value of V_{MIN}CO_{2new} as shown below.

Predicted value of V_{MIN}CO_{2new} is based upon calculation of V_{E}CO₂ at a new setting that may lead to a new V_{T}. This can be done in different ways according to various embodiments of the invention. One way is to calculate the change in V_{MIN}CO₂ by multiplying a tentative change in V_{T} by the fraction of CO₂ in end tidal gas. This way is sufficiently accurate when the alveolar gas has a near constant CO₂ content indicated by a flat alveolar plateau in the SBT-CO₂. At small changes in tidal volume this simple way to calculate a change in V_{E}CO₂ after ventilator resetting is adequate even at modestly sloping alveolar plateau. When the slope of the alveolar plateau and a tentative change in V_{T} is more important, an embodiment of the invention providing more accurate calculation of V_{E}CO₂ at an alternative V_{T} is preferred. The course of CO₂ content of alveolar gas during expiration is reflected in the alveolar plateau of the SBT-CO₂, which is the section after airway gas has been fully expired. The SBT-CO₂ has two formats for presentation, FIG. 3, upper and lower panels. Notably, the basic information in these is the same. In the following example, the alveolar segment of the SBT-CO₂ is considered to be the expiratory curve in both panels after expiration of a volume twice as large as V_{Daw}, in the example 200 ml.

V_{E}CO₂ varies with expired volume, FIG. 3 lower panel. The alveolar segment of the illustrated curve recorded in the ARDS patient could very accurately be described as:

V_{E}CO₂=f(V_{E})=3.17+0.0386·(V_{E}−200)+3.38·10⁻⁵·(V_{E}−200)² Eq. 2

Eq. 2 is just an example of possible ways to mathematically describe the curve for the purpose of the invention. At ventilator resetting, a change of V_{T} will lead to a new value of V_{E}CO₂, V_{E}CO_{2new} that is calculated from Eq. 2 by replacing V_{E} with the new V_{T}, V_{Tnew}.

V_{E}CO_{2new}=3.17+0.0386·(V_{Tnew}−200)+3.38·10⁻⁵·(V_{Tnew}−200)² Eq. 3

In order to calculate V_{T}CO₂, V_{I}CO₂ is subtracted from V_{E}CO₂. V_{I}CO₂ at current ventilator setting is measured as area 28 in FIG. 3. At a change of V_{T}, V_{I}CO₂ will change. V_{I}CO₂ is in general proportional to F_{eT}CO₂.

V_{I}CO_{2new}=V_{I}CO_{2current}·F_{eT}CO_{2new}/F_{eT}CO_{2current} Eq. 4

At a new V_{T}, F_{eT}CO_{2new} is for V_{T} values above 2 times V_{Daw} very accurately derived from SBT-CO₂ in the format shown in FIG. 3 upper panel. In the example:

F_{eT}CO_{2new}=3.74+0.0112·(V_{E}−200)−0.0000222·(V_{E}−200)² Eq. 5

Several alternative mathematical models to describe CO₂ elimination during late part of expiration can be applied as alternatives to Eq. 3 and 5. V_{I}CO₂ is under most circumstances a small fraction of V_{E}CO₂ and varies only slightly with tidal volume due to modest slope of the alveolar plateau as in FIG. 3, upper panel. According to an alternative embodiment of the invention, variation of V_{T} leads to such a small change in V_{I}CO₂ that this change is neglected. In embodiments characterized by that CO₂ is not measured at the y-piece 8 but in the expiratory line 11, V_{I}CO₂ is estimated using the simple algorithm above described. However, in a preferred embodiment the alveolar plateau is described mathematically, Eq. 5, allowing more accurate estimation of V_{I}CO₂, Eq. 4.

Prediction of V_{T}CO₂ after resetting is according to a preferred embodiment of the invention based upon Eq. 3 and 4.

V_{T}CO_{2new}=V_{E}CO_{2new}−V_{I}CO_{2new} Eq. 6

A further factor that influences V_{T}CO₂ is the pattern of inspiration described by the mean distribution time, MDT, and end inspiratory flow, EIF. MDT and EIF vary with RR, T_{I} and T_{P} as shown by Aboab et al.² According to a preferred embodiment of the invention the effect of MDT and EIF is taken into account by using an equation that describes the change in either V_{T}CO₂ or V_{E}CO₂ related to the pattern of inspiration. In ARDS one may e.g. apply the coefficients a, b and c reported in the referred article.

ΔV_{T}CO₂%=a×InMDT+b×EIF+c Eq. 7

Individual coefficients describing the influence of inspiratory pattern on V_{T}CO₂ can according to a preferred embodiment of the invention be measured as described by Aboab et al.² During a period of time, e.g. 1-2 minutes, the pattern of inspiration is changed for a number of breaths, preferably automatically with an apparatus in which the computer may change the pattern. The values of a, b and c are statistically calculated from observations of V_{T}CO₂ or V_{E}CO₂.

By combining Eq. 6 and 7 V_{T}CO_{2new} can be calculated with accuracy enhanced compared to Eq. 6 only:

V_{T}CO_{2new}=f(V_{E}CO_{2new},V_{I}CO_{2new},InMDT,EIF) Eq. 8

Essential for the present invention is that V_{T}CO_{2new} denotes the volume of CO₂ eliminated during some breaths immediately after ventilator resetting. During the following minutes, V_{T}CO₂ will slowly return towards a new steady state defined by the rate of metabolic CO₂ production in ml/min divided by RR.

The product of V_{T}CO_{2new} and RR after resetting, RR_{new}, will give VₘᵢₙCO_{2new}.

V_{MIN}CO_{2new}=RR_{new}·V_{T}CO_{2new} Eq. 9

According to Eq. 1, after ventilator resetting PaCO_{2new} may be predicted from a change in V_{MIN}CO₂. Conversely, in order to achieve a change from the current value PaCO₂ to the new steady state goal value, PaCO_{2goal}, one must reset the ventilator so that:

V_{MIN}CO_{2new}=V_{MIN}CO_{2current}·(PaCO_{2current}/PaCO_{2goal}) Eq. 10

In Eq. 10 the quotient PaCO_{2current}/PaCO_{2goal} may be replaced by a number equal to: 100/(100×X) in which X is how many percent PaCO₂ should decrease to reach the goal. This alternative is applied e.g. when the actual value of PaCO₂ is not known. Furthermore, as explained below, Eq. 10 may be replaced by an equation based upon current and goal values of arterial pH instead of PaCO₂.

According to a preferred embodiment of the invention V_{MIN}CO_{2new} is calculated from current measured values V_{T}CO₂ and PaCO₂ and from PaCO_{2goal}. V_{T}CO_{2new} is for alternative values of V_{T} calculated according to Eq. 2, 3 and 5. For each examined value of V_{Tnew}, RR_{new} is calculated by inserting VₘᵢₙCO_{2new} and V_{T}CO_{2new} in Eq. 9.

In alternative embodiments of the invention the equations can be applied in different order. For example, after calculation of VₘᵢₙCO_{2new} according to Eq. 10, one may for different values of RR calculate V_{T}CO_{2new} from Eq. 9 and then V_{Tnew} from Eq. 3, 4 and 5.

New values of RR imply that the values of MDT and EIF used in the first round of calculations are no longer valid. New values for MDT and EIF, calculated from new values of RR, T_{I} and T_{P}, are entered into Eq. 7 in a second round of calculations. A single iteration is applied in a preferred embodiment of the invention.

With the equations 3-10, the computer can at various modes of ventilation calculate all combinations of values for V_{T}, RR and PaCO₂. It should be observed that minute ventilation is the product of V_{T} and RR. In some ventilators V_{T} and RR are primary parameters, which can be set on the ventilator, implying that minute ventilation, V_{MIN}, is a secondary parameter that follows from values of V_{T} and RR. In other ventilators, V_{MIN} and RR are primary parameters from which V_{T} follows. Throughout this document, what is said about combinations of V_{T} and RR can be transformed to combinations of V_{MIN} and RR.

### Analysis of Respiratory Mechanics

According to a preferred embodiment of the invention, analysis of respiratory mechanics at current and alternative ventilator settings complements the analysis of CO₂ exchange. The analysis of mechanics is based upon recording of airway pressure, P_{AW}, and airway flow rate, F_{AW}. These recordings used to characterize the mechanics are either performed at current ventilator setting or during a procedure in which ventilator operation is modified for the purpose of a more detailed analysis of respiratory mechanics. Expired and inspired volumes, e.g. V_{T}, are derived by integration over time of airway flow rate. Analysis of mechanics serves to minimize or eliminate adverse effects of ventilation. Such effects vary importantly between different patient categories.

In ARDS:
1. Ventilation should maintain PaCO₂, alternatively pH at predefined goal value or goal.
2. V_{T} should be minimal in order to minimize lung trauma due to tidal closure and re-opening of lung units and to permit ventilation at less traumatic airway pressure.
3. P_{PLAT} should be within safe limits so as not to cause hyperdistension or barotrauma.
4. PEEP should be high enough to avoid collapse of lung units during expiration and to maintain an open lung so as to provide adequate conditions for blood oxygenation.

Upon analysis of CO₂ exchange as described, one or more combinations of V_{T} and RR are identified. According to a preferred embodiment of the invention, analysis of further parameters influencing P_{PLAT} and PEEP follows. In the following, a principle according to a preferred embodiment of the invention is described. This principle is based upon the concept that a level of P_{PLAT} that is high but safe with respect to hyperdistension and barotrauma can be defined. 30 cmH₂O is in ARDS a frequently preferred level of P_{PLAT}. In patients with perturbed circulation a lower level may be preferred. In patients with high abdominal and intrathoracic pressure a higher level may be preferred in order to maintain proper lung recruitment.

According to a preferred embodiment of the invention, the analysis of mechanics is performed in parallel with the analysis of CO₂ exchange. Mechanics in terms of elastic and resistive properties of the respiratory system can be derived from measured airway flow rate and pressure according to well known algorithms. Basic elastic property of the respiratory system is expressed as compliance.

Compliance=V_{T}/(P_{PLAT}−PEEP_{TOT}) Eq. 11

PEEP_{TOT} is total PEEP and is the sum of PEEP set and controlled by the ventilator and the pressure that drives flow through the airways at the end of expiration. PEEP_{TOT} is measured during a postinspiratory pause or is estimated according to a previously known algorithm e.g. as described by Jonson et al.³ The elastic properties may be characterized in greater detail, e.g. by studying the elastic pressure volume diagram that can be done with a computer controlled ventilator⁴. Such an expansion of the method is merited if a wide range of lung volume is explored over which compliance varies importantly. An alternative is to avoid drastic ventilator resetting and rather to reset stepwise. After each moderate step, new measurements of SBT-CO₂ and mechanics are done after a period of stabilization to a new steady state.

Inspiratory resistance is incorporated in the analysis for prediction of peak inspiratory upper airway pressure at volume controlled ventilation and in prediction of V_{T} at pressure controlled ventilation in case of inspiratory time too short for establishment of nearly zero end-inspiratory flow rate. Expiratory resistance and compliance may be used for prediction of PEEP_{TOT} according to known algorithms.

### Example of Resetting Guidance According to the Invention

Modern strategies for lung protective ventilation in ARDS and some other conditions are based upon low V_{T}. Then, V_{T} shall be low expressed in relation to body size or its surrogate, which is the so called ideal body weight. According to a preferred embodiment of the invention, V_{T} is then replaced or paralleled by V_{T}/kg in mentioned calculations. The example below refers to data from the ARDS patient represented by FIG. 3 ventilated under volume control. The values of coefficients a-c used in Eq. 6 were average values in ARDS patients according to Aboab et al.²

The operator starts the process Resetting Guidance. The operator selects ARDS from a list of different diagnoses. Using data from the ARDS patient and the equations 2-11 an embodiment of the invention is illustrated with the following example illustrating a preferred embodiment of the invention when used by an operator with ordinary experience.

The computer returns current ventilator setting and default values representing physiological goals and such limits of parameters defining the mode of operation, which are recommended for the particular patient category at the intensive care unit in question. The operator may accept or change these Goals and Limits, which are shown in Table 1. The default goals were: Unchanged or lower PaCO₂ and V_{T}=6 ml/kg. The default values for T_{I} and T_{P} were 15 and 28% of the respiratory cycle, respectively, leaving the relative time for expiration at 57% unchanged, which represent current knowledge about CO₂ exchange in ARDS.

TABLE 1

| | Current | Goals | |
|---|---|---|---|
| | Settings and | and | |
| | Observations | limits | Solution |
| PaCO₂ mmHg | 58 | ≦58 | 57.5 |
| V_{T}, ml/kg | 7.1 | 6.0 | 6.0 |
| V_{T} ml | 392 | 330 | 330 |
| RR/minut | 22 | ≦30 | 29 |
| T_{I} % | 33 | 15 | 15 |
| T_{P} % | 10 | 28 | 28 |
| PEEP cmH₂O | 15 | | 13.5 |
| P_{PLAT} cmH₂O | 35 | 30 | 30 |
| Compliance, ml/cmH₂O | 20 | | |

In the example default values were accepted whereupon the computer returned a Solution based upon Eq. 2-10 for CO₂ exchange and solving Eq. 11 for PEEP_{TOT}. All Goals and Limits could be met as shown in Table 1. Predicted PaCO₂ was not significantly lower than current.

According to a preferred embodiment of the invention, in which the computer may control the ventilator, the operator can accept the Solution, which is then automatically implemented. In alternative types of systems he resets the ventilator manually according to the Solution.

If all goals cannot be achieved under the defined limits, the computer highlights the problem. Then, the operator enters alternative values for Goals and/or Limits to get new guidance from a valid solution.

According to a preferred embodiment of the invention, highly experienced operators are offered a larger degree of freedom to choose a combination of settings predicted to lead to the goals. In the example illustrated in Table 2 the operator required solutions based upon the goals that PaCO₂ should be reduced from 58 to 54 mmHg, V_{T} should be ≦6 ml/kg and RR≦60 min⁻¹, T_{I}=0.2 and T_{P}=0.3. The computer returned the solutions in Table 2.

TABLE 2

| | Current | Goals | | | | | | | | | |
|---|---|---|---|---|---|---|---|---|---|---|---|
| | Settings and | and | Solutions | | | | | | | | |
| | Observations | Limits | 1 | 2 | 3 | 4 | 5 | 6 | 7 | 8 | 9 |
| PaCO₂ | 58 | 54 | 54 | 54 | 54 | 54 | 54 | 54 | 54 | 54 | 54 |
| mmHg | | | | | | | | | | | |
| V_{T} ml/kg | 7.1 | ≦6 | 6.0 | 5.8 | 5.6 | 5.5 | 5.3 | 5.2 | 5.0 | 4.8 | 4.7 |
| RR min⁻¹ | 22 | ≦60 | 31 | 34 | 36 | 39 | 42 | 45 | 48 | 52 | 56 |
| V_{T} ml | 392 | — | 330 | 320 | 310 | 301 | 292 | 283 | 275 | 267 | 259 |
| T_{I} % | 33 | 0.2 | 0.2 | 0.2 | 0.2 | 0.2 | 0.2 | 0.2 | 0.2 | 0.2 | 0.2 |
| T_{P} % | 10 | 0.3 | 0.3 | 0.3 | 0.3 | 0.3 | 0.3 | 0.3 | 0.3 | 0.3 | 0.3 |
| PEEP | 15 | | 13.5 | 14.0 | 14.5 | 15.0 | 15.5 | 16.0 | 16.0 | 16.5 | 17.0 |
| cmH₂O | | | | | | | | | | | |
| P_{PLAT} | 35 | 30 | 30 | 30 | 30 | 30 | 30 | 30 | 30 | 30 | 30 |
| cmH₂O | | | | | | | | | | | |
| Compliance | 20 | | | | | | | | | | |
| ml/cmH₂O | | | | | | | | | | | |

The operator judges the solutions in Table 2 and may choose one that is implemented automatically or manually depending upon facilities offered by the properties of the apparatus. The higher RR he is ready to accept, the lower V_{T} will be and the higher PEEP will be. Although lung protection would be optimal at Solution 9 the operator might consider that a change of RR from 22 to 57 min⁻¹ is too drastic for accurate computer prediction of outcome. He can then choose one of the less radical solutions and perform a new test later for a second step of resetting. If he judges that no satisfactory solution is presented, he might adjust Goals and Limits in order to explore other combinations of settings.

Lov V_{T} is a most important means behind lung protective ventilation. Accordingly the operator may wish to use a particular value for V_{T} as a fixed goal from which the system predicts which values of PaCO₂ are to be expected at different values of RR. Table 3 shows an example based upon the same patient as in Table 2. The operator has chosen to analyse a V_{T} value of 5.5 ml/kg. From that value a preliminary value of V_{T}CO_{2new} is calculated according to Eq. 3-8. The latter value multiplied by different values for RR give a series of preliminary values for V_{MIN}CO_{2new}. By iteration the latter values are adjusted for changes in MDT and EIF associated with each RR value. Resulting adjusted values for V_{MIN}CO_{2new} together with measured values for V_{MIN}CO_{2current} and PaCO_{2current} give a series of values for PaCO_{2new} corresponding to analysed values for RR according to Eq. 1. The operator may from Table 3 chose one of the solutions for manual or automatic implementation.

TABLE 3

| | Current | Goals | | | | | | | | | |
|---|---|---|---|---|---|---|---|---|---|---|---|
| | Settings and | and | Solutions | | | | | | | | |
| | Observations | Limits | 1 | 2 | 3 | 4 | 5 | 6 | 7 | 8 | 9 |
| PaCO₂ | 58 | <60 | 59 | 54 | 51 | 48 | 45 | 42 | 40 | 38 | 37 |
| mmHg | | | | | | | | | | | |
| V_{T} ml/kg | 7.1 | 5.5 | 5.5 | 5.5 | 5.5 | 5.5 | 5.5 | 5.5 | 5.5 | 5.5 | 5.5 |
| RR min⁻¹ | 22 | <60 | 33 | 36 | 39 | 42 | 45 | 48 | 51 | 54 | 57 |
| V_{T} ml | 392 | — | 301 | 301 | 301 | 301 | 301 | 301 | 301 | 301 | 301 |
| T_{I} % | 33 | 20 | 20 | 20 | 20 | 20 | 20 | 20 | 20 | 20 | 20 |
| T_{P} % | 10 | 30 | 30 | 30 | 30 | 30 | 30 | 30 | 30 | 30 | 30 |
| PEEP | 15 | | 14.5 | 14.5 | 14.5 | 14.5 | 14.5 | 14.5 | 14.5 | 14.5 | 14.5 |
| cmH₂O | | | | | | | | | | | |
| P_{PLAT} | 35 | 30 | 30 | 30 | 30 | 30 | 30 | 30 | 30 | 30 | 30 |
| cmH₂O | | | | | | | | | | | |
| Compliance | 20 | | | | | | | | | | |
| ml/cmH₂O | | | | | | | | | | | |

According to alternative embodiments of the invention, Goals and Limits as well as Solutions may be expressed in other ways than illustrated above. Accordingly, the invention allows wide variations in set up of the system with respect to formulation of Goals and Limits, presentation of results and how to implement the results. For example, rather than to choose a value of PaCO₂ as a goal one may use arterial pH. Values of pH, PaCO₂ and complimentary acid/base data comprised in a routine clinical acid/base status allow transformation of a goal pH to a goal PaCO₂ and vice versa using well known algorithms.

As stated above, when the invention is practiced in a system in which the computer performing the calculations has no means to control the ventilator, resetting of the ventilator is left to the operator. At volume controlled ventilation he sets the ventilator in accordance with results of the calculations. When the invention is practiced in a system in which the computer performing the calculations has means to control the ventilator, a new mode of operation which accords with the calculations may be automatically implemented.

Eq. 2-11 are valid at all modes of ventilation. However, direct implementation of the results in terms of new values for V_{T} and RR is possible only at volume controlled ventilation. At other modes indirect methods to reach these values are needed. For example, at pressure controlled ventilation, a new RR but not a new V_{T} can be directly set on the ventilator. Analysis of respiratory mechanics widens the scope of application of the invention. As an example, according to an embodiment of the invention the computer transforms a new value of V_{T} to a difference between inspiration pressure and PEEP such that V_{T} reaches its goal value at the current value of respiratory system compliance. This principle can be practiced in several ways. Inspiratory pressure, P_{INSP}, or PEEP can be modified according to the equation:

V_{Tnew}=Compliance/(P_{INSP}−PEEP) Eq. 12

After resetting RR to a new value, an alternative is to stepwise modify P_{INSP} or PEEP until the new value of V_{T} is attained. This can be done manually. When the computer 17 or 20 can control the function of the ventilator, a preferred embodiment of the invention permits changes of pressure values stepwise until the goal value of V_{T} is achieved.

It is in the nature of modes for spontaneous supported ventilation that the patient is free to influence the ventilation. Nevertheless, an analysis along the principles described is valid as is identification of V_{T}, RR and other parameters describing features of ventilation, which would lead to specified goals. Implementation of such features cannot be done directly as for controlled modes of ventilation. Methods for implementation must be adapted to how the supported mode functions and there are many varieties of support in different types of ventilators. Only an outline of pressure support is given here. In pressure support mode adjustment of P_{PLAT} can be done by modifying the inspiratory pressure. By increasing PEEP the tidal volume may be reduced. Secondary to a lower V_{T}, RR will increase due to the ordinary physiological control of spontaneous ventilation.

When the invention is practiced in context with Neurally Adjusted Ventilatory Assist, NAVA, ventilation is highly influenced by the efforts of the patient. The amplification from the diaphragm EMG to the pressure control of the ventilator is according to an embodiment of the invention slowly modified until the goal value of tidal volume is reached. The respiratory centre of the patient will then in accordance with the principles behind NAVA adjust RR to maintain adequate control of PaCO₂ or pH.

When a ventilator is drastically reset, predictions of the outcome are less reliable than after a more modest resetting. According to a preferred embodiment of the invention, far-reaching changes of ventilator settings are avoided by warnings and or limitations, particularly in such algorithms practiced by less experienced operators. Rather than allowing sudden large changes in ventilator settings the system then suggests a limited resetting and a repeated process of measurement, calculations and resetting. This should be made after a period long enough to establish a new steady state, i.e. 15-30 minutes. Such stepwise resetting leads to more accurately optimised ventilation of the patient.

At steady state, PaCO₂ is proportional to the quotient between metabolic CO₂ production and alveolar ventilation. A change in metabolic rate will affect PaCO₂ thereby leading to deviation from the goal defined by the operator. Such a change will affect measured V_{MIN}CO₂ values. Changes in measured values of V_{MIN}CO₂ over longer periods, e.g. 30 minutes, which are not related to changes in ventilation, indicate variations in metabolic CO₂ production. According to a preferred embodiment of the invention such changes are detected and reported by the apparatus in conjunction with follow up reports after ventilator resetting like in the following example:

V_{MIN}CO₂ increased by 15% over 60 minutes. This indicates increased metabolic rate!

Notably, the described analysis of CO₂ exchange is applicable in all types of patients although goals may differ. In ARDS normocapnia or moderate hypercapnia is preferred. At brain trauma hypocapnia is often a goal together with low mean airway pressure. In COPD exacerbation, correction of excessive values of PaCO₂ or pH and reduction of high P_{PLAT} values leading to hyperdistension are rational goals.

The example of the invention illustrated in Table 2 adheres to the principle for mechanical ventilation in ARDS depicted in the outlined by Uttman et al.⁵ According to this principle the lowest possible V_{T} compatible with adequate CO₂ exchange should be applied in combination with a high but safe P_{PLAT}. This leads to highest possible PEEP under the circumstances. High PEEP will maintain lung recruitment and optimal condition for blood oxygenation at low or moderate levels of F_{I}O₂. When the invention is applied in this way, F_{I}O₂ is adjusted to a level that maintains the goal with respect to PaO₂ or S_{P}O₂.

An alternative way to apply the invention accords with ARDSnet recommendations. Then, adequate blood oxygenation is achieved by choosing a combination of PEEP and F_{I}O₂ while combinations of V_{T} and RR satisfying the goals are determined as described above. If no solution is identified that fulfils the goal with respect to P_{PLAT} (Eq. 11) this is notified as a guideline for the operator who may analyze an alternative combination of goals and limitations before ventilator resetting. The invention can furthermore be adapted to different strategies in various patient populations in dependence upon growing knowledge and progress in the field.

Going back to the chain of calculation expressed in Eq. 1-11 the invention is based upon that CO₂ exchange, PaCO₂ and pH are determined by a combination of values for V_{T}, RR and pattern of inspiration, together with the characteristics of the SBT-CO₂. Any combination of PaCO₂, V_{T} and RR can be explored. Furthermore, alveolar pressure that during breaths alternates between PEEP_{TOT} and P_{PLAT}, depends on V_{T} and PEEP together with the characteristics of elastic pressure/volume properties. The latter can be expressed in terms of a value of compliance or in the format of an elastic pressure volume curve that takes non-linear elastic properties into account. Hence, it is possible to analyse all feasible combinations of either PaCO₂ or pH, V_{T}, RR and either P_{PLAT} or PEEP in a search for a ventilator resetting that is regarded as optimal in any patient category. The influence on PEEP_{TOT} by auto-PEEP can furthermore be explored on the basis of values for V_{T}, RR, compliance and resistance of the respiratory system. Such an expansion of the calculations is merited in COPD patients but also in other patient categories when high values of RR are explored. At pressure controlled ventilation also the inspiratory time constant of the respiratory system can be taken into account as a factor influencing V_{T}. Embodiments incorporating calculation of inspiratory resistance allow prediction also of peak airway pressure. Predictions of outcome according to what has been stated do not guarantee that the results and solutions will be in full agreement with the true outcome of a resetting. Therefore, feedback and follow up is an important feature.

### Feedback and Immediate Follow Up of Ventilator Resetting

After ventilator resetting an immediate feedback indicating to what extent the resetting is leading towards defined goals is presented. According to a preferred embodiment of the invention this is based upon principles described in Swedish patent application, SE1200155-8. With respect to lung mechanics, feedback is based upon direct measurements of e.g. V_{T}, RR, P_{PLAT} and PEEP. With respect to PaCO₂ or pH, prediction is based upon preceding values of PaCO₂ and V_{MIN}CO₂, i.e. PaCO_{2current} and V_{MIN}CO_{2current} together with V_{MIN}CO_{2new} that in this context is determined immediately after resetting. From Eq. 10 follows:

PaCO_{2new}=V_{MIN}CO_{2current}/V_{MIN}CO_{2new}·PaCO_{2current} Eq. 13

Within some breaths after resetting, the results of the resetting are presented. When measured values significantly differ from goals an alarm is raised. According to a preferred embodiment of the invention, the precedent setting is re-instituted either manually or automatically. Deviations between measured values and goals may be due to that resetting was too far-reaching. The computer may in such a case propose a more moderate resetting. Notably, if follow up and reaction to the follow up is achieved within a couple of minutes, the CO₂ stores in the body have not been brought out of steady state and a new resetting procedure may be undertaken without further delay.

At modest differences between predicted data and data measured immediately after resetting, corrections of settings may be performed, either automatically in case of computer control of the ventilator, or else manually. As influences on PEEP_{TOT} are complex, adjustment of set PEEP may often be indicated in order to achieve the goal for P_{PLAT} or total PEEP. Minor differences between goal and data measured immediately after resetting are expected and may be neglected.

Final achievement of the predicted PaCO₂ value can be checked by analysis of an arterial blood sample after a period of steady state establishment. The computer can at that time inform about significant changes in CO₂ elimination, which may indicate a change in metabolic rate. After a new blood gas test a second procedure for optimisation of ventilator setting may be considered.

Multiple Step Ventilator Resetting—Automated Closed Loop Ventilation

According to a preferred embodiment of the invention, a computer that can control the ventilator is programmed to reach and maintain goals specified by the operator by repeated ventilator resetting within limits defined by the operator or by default values for a particular category of patients. According to a preferred embodiment of the invention, the procedure starts with a presentation of solutions satisfying Goals and Limits like that in Table 2 in which V_{T} is reduced by 3% for each step from solution 1 to 9. In a preferred embodiment of the invention, computer controlled automatic changes of ventilator setting are performed at time intervals sufficiently long for establishment of a steady state. Furthermore, the extent of change at each step that is not supervised by an attending operator is limited in order to avoid resetting for which predicted outcome may be less accurate and against which the patient may show intolerance. Using Table 2 as an example and assuming that solution 1 was chosen as a first step, and that the immediate outcome of that step was found adequate by the supervising operator; Multiple step ventilator resetting—Automated closed loop ventilation may be activated. If a solution representing a final specified Goal, e.g. number 9 is chosen together with a maximum V_{T} change of 6% per step, the computer would change ventilator setting according to step 3, 6 and 9, at defined time intervals. Each resetting is preceded by measurement and analysis so as to more accurately define which setting of for example RR and PEEP is required to reach a following step of V_{T} reduction. Immediately after each step the computer examines the outcome. At small deviations from specified goals for example with respect to P_{PLAT}, the computer executes correction of PEEP to reach the goal. If goals are achieved after the final resetting according to solution 9 in Table 2, the computer performs regular tests followed by correction of ventilator setting if needed to maintain a status according to the goals.

At computer controlled automatic changes of ventilator setting, continuous meticulous monitoring of the procedure and status of the patient is essential. According to a preferred embodiment of the invention the computer automatically supervises not only the signals from transducers shown in FIGS. 1 and 2, but also signals representing oxygenation and circulation. When monitoring indicates a patient status outside set limits, further resetting of the ventilator is cancelled and an alarm is issued.

### REFERENCES

1. Ventilation with lower tidal volumes as compared with traditional tidal volumes for acute lung injury and the acute respiratory distress syndrome. The Acute Respiratory Distress Syndrome Network. N Engl J Med 2000, 342:1301-1308.
2. Aboab J, Niklason L, Uttman L, Brochard L, Jonson B: Dead space and CO2 elimination related to pattern of inspiratory gas delivery in ARDS patients. Crit Care 2012, 16:R39.
3. Jonson B, Nordstrom L, Olsson S G, Akerback D: Monitoring of ventilation and lung mechanics during automatic ventilation. A new device. Bull Physiopathol Respir (Nancy) 1975, 11:729-743.
4. Bitzen U, Enoksson J, Uttman L, Niklason L, Johansson L, Jonson B: Multiple pressure-volume loops recorded with sinusoidal low flow in a porcine acute respiratory distress syndrome model. Clin Physiol Funct Imaging 2006, 26:113-119.
5. Uttman L, Bitzen U, De Robertis E, Enoksson J, Johansson L, Jonson B: Protective ventilation in experimental acute respiratory distress syndrome after ventilator-induced lung injury: a randomized controlled trial. Br J Anaesth 2012.

1. A system for mechanical ventilation comprising:
transducers for measurement of flow rate and CO₂; and a computer that records and analyses the transducer signals according to the principle of volumetric capnography, wherein the computer is programmed for analytic mathematical analysis of data recorded before a ventilator resetting in order to identify one or more alternative combinations of values describing a mode of ventilator operation, which combinations comprise at least tidal volume and respiratory frequency, and which are predicted to lead to achievement of specified goals representing one or more of the parameters comprising arterial partial pressure of CO₂, arterial pH and tidal volume, which identification is performed with calculations based upon a measured volume of CO₂ eliminated per minute or other unit of time, a measured content of CO₂ in expired alveolar gas and a change of volume of CO₂ eliminated per breath that a change of current tidal volume is calculated to bring about.

2. A system for mechanical ventilation according to claim 1, wherein the calculation of CO₂ volume eliminated per unit time after ventilator resetting is based upon data measured before ventilator resetting describing a course of CO₂ content of alveolar gas during expiration.

3. A system for mechanical ventilation according to claim 1, wherein the computer is further programmed to analyse signals for flow rate and pressure with respect to mechanical properties of a respiratory system and thereby to identify at least one ventilator setting, which on the basis of an identified mode of operation characterised by a particular tidal volume is predicted to lead to a specific goal with regard to post-inspiratory plateau pressure.

4. A system for mechanical ventilation according to claim 1, wherein the computer is further programmed to analyse signals for flow rate and pressure with respect to mechanical properties of a respiratory system and thereby to identify at least one ventilator setting, which on the basis of an identified mode of operation characterised by a particular tidal volume is predicted to lead to a specific goal with regard to positive end expiratory pressure.

5. A system for mechanical ventilation according to claim 1, wherein the computer is further programmed to identify at least one ventilator setting leading to specified goals with respect to minimal adverse effects of ventilation comprising a combination of the parameter tidal volume and one of the parameters comprising post-inspiratory plateau pressure and positive end-expiratory pressure.

6. A system for mechanical ventilation according to claim 1, wherein the computer is further programmed to identify more than one combination of parameters describing the mode of ventilator operation, which stepwise are predicted to lead towards specified goals as guidance for the operator.

7. A system for mechanical ventilation according to claim 1, wherein the computer is configured for controlling the ventilator, wherein the computer is so programmed that a current mode of ventilator operation is automatically substituted by a new mode of operation leading to specified goals.

8. A system for mechanical ventilation according to claim, wherein the computer is further programmed to substitute in more than one step the current mode of operation by new modes of operation which stepwise lead towards specified goals.

9. A system for mechanical ventilation according to claim 1, wherein the computer is further programmed to measure an outcome of ventilator resetting within a few breaths after resetting, and to report about the outcome and to issue an alarm if specified goals are not appropriately approached.

10. A system for mechanical ventilation according to claim 1, wherein the computer is further programmed to perform automated multiple measurements and automated resetting to reach and maintain specified goals.

11. A system for mechanical ventilation according to claim 1, wherein the computer is further programmed to perform a series of test breaths having a varying pattern of inspiration and from resulting volumes of CO₂ exchanged during individual test breaths, mathematically characterise how the pattern of inspiration affects the exchange of CO₂ and from this analysis predict which is an optimal pattern of inspiration for reaching specified goals.
